(19) United States
(12) Patent Application Publication
(10) Pub. No.: US 2024/0042088 A1
(43) Pub. Date: Feb. 8, 2024

(54) Multi-Chambered Ultraviolet Air Sterilizer and Purifier

(71) Applicant: CalyxPure, Inc., Houston, TX (US)

(72) Inventors: John C. Higgins, Houston, TX (US); Mark Sam, Bellaire, TX (US); James Higgins, Conroe, TX (US); Jonathan Evans, Conroe, TX (US)

(21) Appl. No.: 17/816,931

(22) Filed: Aug. 2, 2022

(51) Int. Cl. A61L 9/20; A61L 9/12

(52) U.S. Cl. CPC A61L 9/205; A61L 9/122; A61L 2209/14; A61L 2202/25; A61L 2209/111

(57) ABSTRACT

A sterilization box for treating room air is described. The air drawn into the sterilization box is irradiated with UV radiation, such as is provided from UV LED chips. The air may be drawn into the sterilization box by under-pressure within the box created by fans at the exit ports of the box. The air may be drawn into a first chamber of the box where it is treated with one wavelength of UV radiation and then passed to a second chamber, where it is treated with a second wavelength of UV radiation. After the air is sterilized, it can be put back into the room or building in which the fixture is placed.

### FIELD OF THE INVENTION

This application relates to an apparatus having the ability to sterilize pathogens such as bacteria, fungi, and viruses and to oxidize volatile organic compounds (VOCs) in room air.

### INTRODUCTION

The COVID-19 global health crisis highlights the need for methods and systems for disinfecting pathogens in the air, especially in indoor settings, such as homes, offices, school rooms, and the like. Various systems that use ultraviolet (UV) irradiation to disinfect/deactivate pathogens, such as the novel coronavirus (SARS-CoV-2) that is responsible for COVID-19, have been proposed. Another air quality issue that arises, particularly within industrial settings, is the presence of volatile organic compounds (VOCs). VOCs may be both odorous and harmful to animal and human health. Accordingly, there is a need in the art for air treatment systems capable of disinfecting pathogens and purifying air of VOCs.

### SUMMARY

Disclosed herein is an air sterilization box for treating room air, the sterilization box comprising: an intake chamber configured to receive room air drawn into the sterilization box, a first plurality of first ultraviolet (UV) light emitting diodes (LEDs) within the intake chamber configured to irradiate air drawn into the intake chamber with UV radiation having a first peak wavelength within a first wavelength range, one or more flow paths configured to receive air from the intake chamber, and a second plurality of second UV LEDs within the one or more flow paths configured to irradiate air drawn through the flow paths to produce treated air, wherein the second UV LEDs provide radiation having a second peak wavelength that is different than the first peak wavelength. According to some embodiments, the first UV LEDs are configured to produce UV radiation with a peak wavelength in the range from 315 to 400 nm. According to some embodiments, the intake chamber comprises a photoactive filter comprising titanium dioxide (TiO2) configured to catalyze generation of reactive oxygen species (ROSS) when irradiated with radiation from the first UV LEDs. According to some embodiments, the intake chamber is configured so that the ROSs oxidize volatile organic compounds (VOCs) in air drawn into the intake chamber. According to some embodiments, the chamber comprises one or more windows configured to pass air in the intake chamber into the one or more flow paths. According to some embodiments, the flow paths terminate at an exit port and wherein the sterilization box further comprises a fan at each exit port configured to move treated air out of the sterilization box, thereby creating an under-pressure within the sterilization box. According to some embodiments, each of the one or more flow paths are non-linear. According to some embodiments, each of the flow paths are serpentine or spiral along at least a portion of their lengths. According to some embodiments, each of the flow paths are defined by one or more baffles. According to some embodiments, each of the flow paths have widths that change over the length of the flow path. According to some embodiments, the second peak wavelength in the range from 200 to 280 nm. According to some embodiments, the second UV LEDs are configured to produce the UV radiation capable of sterilizing biological pathogens in the air. According to some embodiments, the sterilization box comprises an interior comprising a reflective coating. According to some embodiments, the reflective coating is photocatalytically active. According to some embodiments, the reflective coating comprises TiO2 crystals. According to some embodiments, the sterilization box further comprises a bottom configured to connect to a light fixture. According to some embodiments, the sterilization box further comprises a bottom configured to connect to a ceiling tile. According to some embodiments, the sterilization box has four flow paths. According to some embodiments, the sterilization box has two flow paths. According to some embodiments, the sterilization box further comprises filters at each of the exit ports configured to filter the treated air as it exits the sterilization box. Any of the UV LEDs may be operated in a continuous mode or may be pulsed.

Also disclosed herein is an air sterilization box for treating room air, the sterilization box comprising: two or more flow paths, each flow path terminating, at an exit port, a fan at each exit port configured to move treated air out of the sterilization box, thereby creating an under-pressure within the sterilization box, an intake chamber configured to receive room air drawn into the sterilization box by the under-pressure and to divide the room air into each of the flow paths, and a plurality of first ultraviolet (UV) light emitting diodes (LEDs) configured to irradiate air with UV radiation as it is drawn through each of the flow paths to produce the treated air. According to some embodiments, each of the flow paths are non-linear. According to some embodiments, each of the flow paths are serpentine or spiral along at least a portion of their lengths. According to some embodiments, each of the flow paths are defined by one or more baffles. According to some embodiments, each of the flow paths have widths that change over the length of the flow path. According to some embodiments, the first UV LEDs are configured to produce the UV radiation with a peak wavelength in the range from 200 to 280 nm. According to some embodiments, the first UV LEDs are configured to produce the UV radiation capable of sterilizing biological pathogens in the air. According to some embodiments, the sterilization box comprises an interior comprising a reflective coating. According to some embodiments, the reflective coating is photocatalytically active. According to some embodiments, the reflective coating comprises titanium dioxide (TiO2) crystals. According to some embodiments, the sterilization box further comprises a bottom configured to connect to a light fixture. According to some embodiments, the sterilization box further comprises a bottom configured to connect to a ceiling tile. According to some embodiments, the sterilization box comprises four flow paths. According to some embodiments, the sterilization box comprises two flow paths. According to some embodiments, the sterilization box further comprises filters at each of the exit ports configured to filter the treated air as it exits the sterilization box. According to some embodiments, the intake chamber comprises second UV LEDs configured to produce UV radiation with a peak wavelength different than that of the first UV LEDs. According to some embodiments, the second UV LEDs are configured to produce UV radiation with a peak wavelength in the range from 315 to 400 nm. According to some embodiments, the intake chamber comprises a photoactive filter comprising TiO2 configured to catalyze generation of reactive oxygen species (ROSs) when irradiated with radiation from the second UV LEDs. According to some embodiments, the intake chamber is configured so that the ROSs oxidize volatile organic compounds (VOCs) in air drawn into the intake chamber. According to some embodiments, the sterilization box further comprises a plurality of third UV LEDs configured to irradiate air in the flow paths, wherein third UV LEDs are configured to produce UV radiation with a peak wavelength different than that of the first UV LEDs. Any of the UV LEDs may be operated in a continuous mode or may be pulsed.

### BRIEF DESCRIPTION OF THE DRAWINGS

FIG. 1 shows various views of an improved lighting fixture, having a light box, a fan to draw in air, and a UV sterilization box through which the drawn air passes.

FIGS. 2A and 2B show white LED chips that can be used in the light box, which preferably produce a white light spectrum having significant near-UV peak wavelengths at 405 and 470 nm, which have shown to be useful to inactivate bacteria and fungi.

FIG. 3A shows a cross section of the lighting fixture, and FIG. 3B shows the fixture's back plane, diffuser, and one of its circuit boards of the light box.

FIG. 4 shows a top down view of the UV sterilization box with its cover removed, including UV LED chips and baffles to define a non-linear path for the air drawn into the fixture by the fan.

FIG. 5 shows the system electronics for the fixture, including the provision of power to the driver circuitries for the white LED chips, the UV LED chips, and the fan.

FIG. 6A shows that sterilized air output from the UV sterilization box can be output back into a room through ports provided in the light box.

FIG. 6B shows that sterilized air outputs from the UV sterilization box can be combined, and FIG. 6C shows how that sterilized air can be output into the air handling system of a building or house.

FIGS. 7A and 7B show a further embodiment of a sterilization box.

FIGS. 8A and 8B show the inside of a further embodiment of a sterilization box.

FIGS. 9A and 9B show an intake chamber of a sterilization box and an input filter, respectively.

FIGS. 10A-10C show views of a baffle insert.

FIG. 11 shows an exploded view of an embodiment of a sterilization box.

FIG. 12 shows air flow paths within an embodiment of a sterilization box.

### DETAILED DESCRIPTION

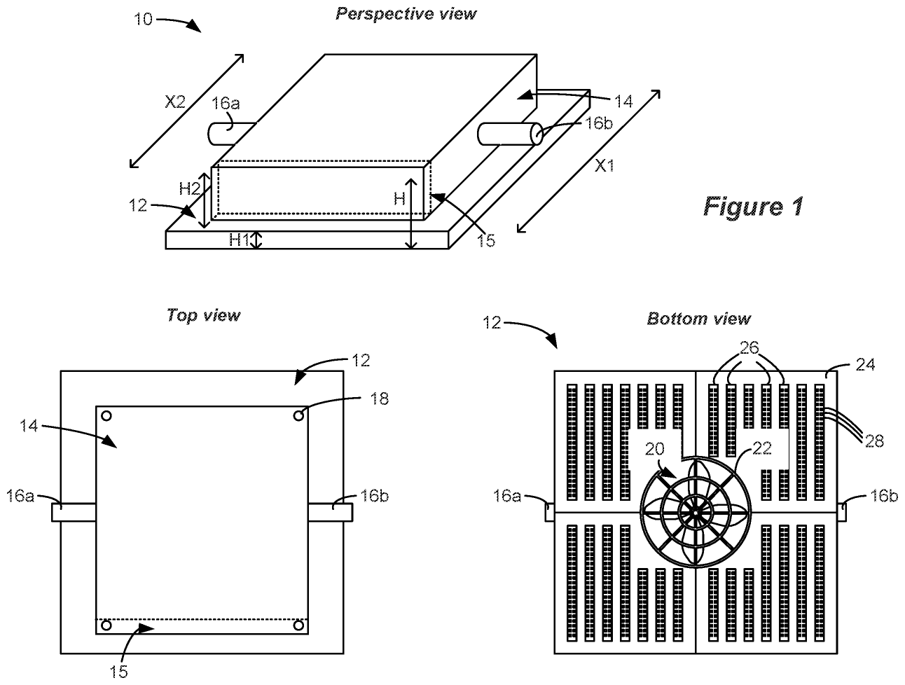

An example of a disinfecting light fixture 10 is shown in FIG. 1 in perspective, top down, and bottom up views. The fixture 10 has two main sections: a light box 12, and a UV sterilization box 14. Note that these “boxes” 12 and 14 do not need to be box-shaped as shown, and boxes 12 and 14 can instead be understood as any compartment, region, or volume in the fixture 10 however shaped and sized.

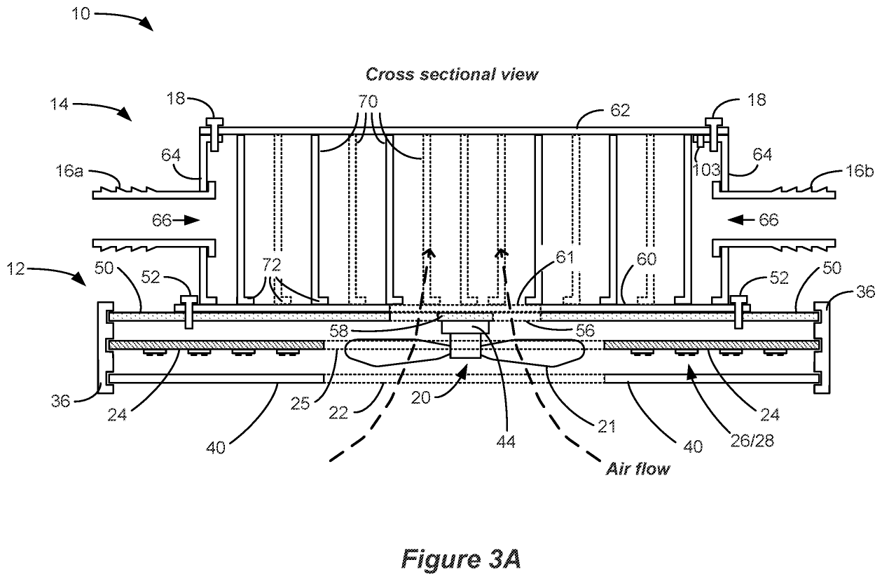

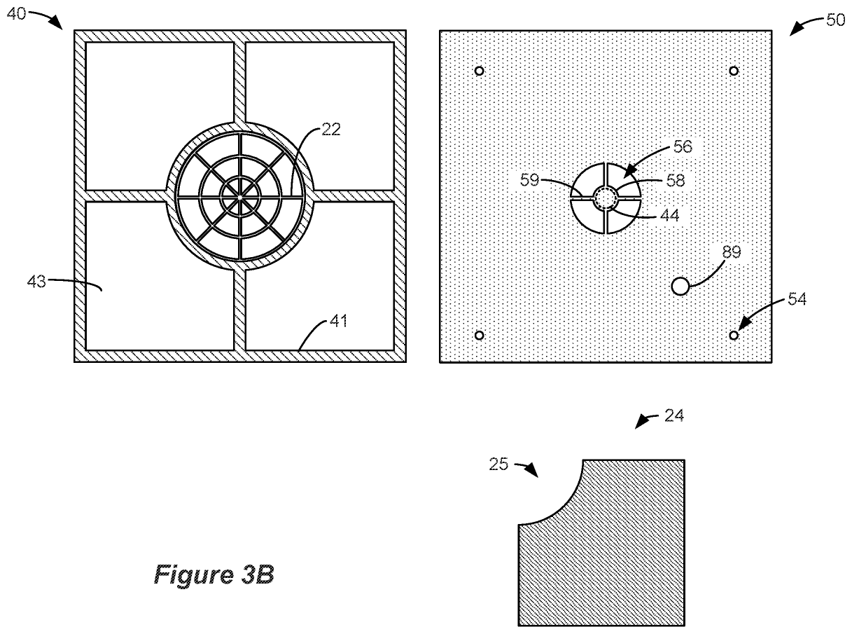

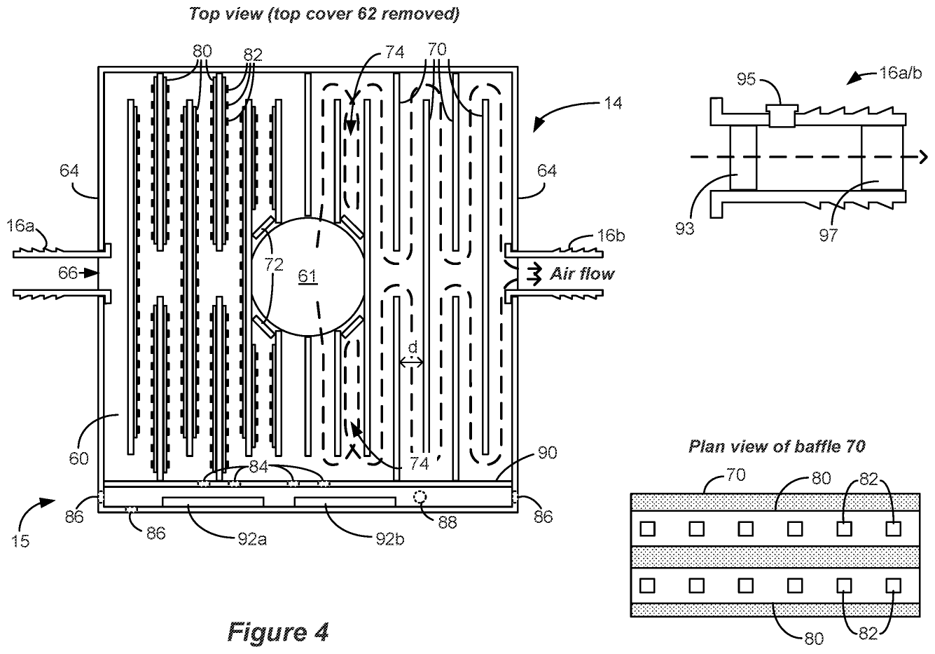

The light box 12 includes white LED chips 28 which provide for illumination and whose spectrum additionally and preferably includes significant radiation at 405 nm and 470 nm, as explained further below. The light box 12 includes a fan 20 protected by a grate 22. The fan is used to draw air into the UV sterilization box 14 where the air is disinfected with UV radiation provided by UV LED chips 82 (FIG. 4), again discussed further below. One or more holes 66 (FIG. 3A) are present in the UV sterilization box 14, and hose connectors 16a and 16b can be fitted in these holes. The air drawn into the UV sterilization box 14 by the fan 20 exits the fixture 10 through these hose connectors 16a and 16, thus outputting sterilized air.

Notice then that the disinfecting light fixture 10 includes different means of providing sterilization of pathogens. The white LED chips 28, as well as providing white light for illumination, include significant radiation at 405 and 470 nm, which are useful in inactivating at least bacteria and fungi in the air and on surfaces in the room being illuminated, as discussed above. Other air borne pathogens—in particular viruses—are drawn into the fixture by the fan 20 and subjected to high intensity UV radiation provided by the UV LED chips 82 in the UV sterilization box 14. Such UV radiation should inactivate such air borne viruses, see C. D. Lytle et al., “Predicted Inactivation of Viruses of Relevance to Biodefense by Solar Radiation,” J. Virology (Vol. 79 (22), pp. 14244-52 (2005), and would be expected to provide further sterilization of other air borne pathogens (bacteria and fungi) as well. The air as sterilized by the fixture 10 can then be put back into the room where the fixture 10 is located, or otherwise may be input into the air handling system of the building, as explained further below. Notice that the fixture 10's sterilization properties makes it particularly well suited for use in locations where pathogens can be problematic, such as hospitals, nursing homes, etc. Fixture 10 is also useful when incorporated into grow light systems use to grow plants, such as in the system described in U.S. Pat. No. 10,440,900 which is incorporated herein by reference in its entirety. Sterilization is important in this context as well, because growing plants are susceptible to pathogens such as viruses, bacteria, and fungi.

FIG. 1 shows an example of a 2×2 feet (X1) fixture 10, although the fixture could be made of any shape and size. The UV sterilization box 14 may be smaller in area, e.g., approximately 1.5×1.5 feet (X2). The total height H of the fixture 10 is preferably about six inches, with the light box 12 having a height of about 1.5 inches (H1) and the UV sterilization box 14 having a height of about 4.5 inches (H2). These dimensions are merely one example, and both the light box 12 and the UV sterilization box 14 can have other dimensions as well. The fixture 10 so sized comprises a suitable replacement for traditional fluorescent bulb fixtures. Means for mounting the fixture 10 (e.g., to a room's ceiling) are not shown, but can be of conventional design.

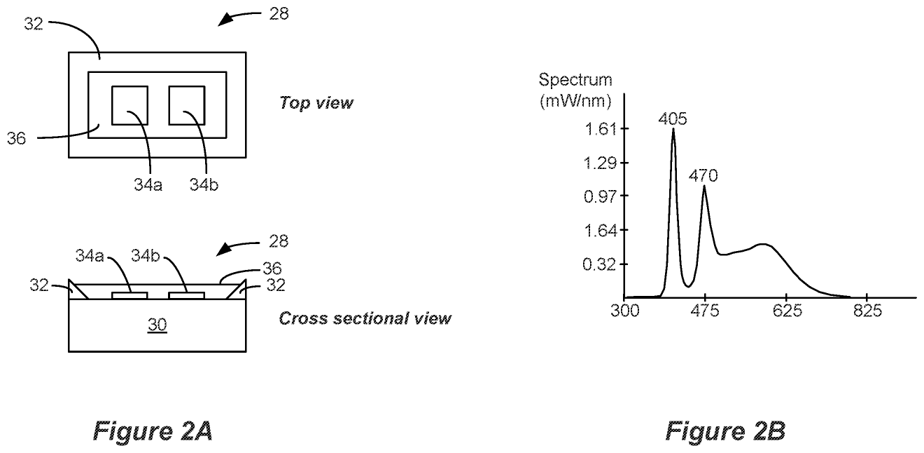

The top view shows that the UV sterilization box 14 can include a section 15 for necessary system electronics, as described later. The bottom view shows the underside of the fixture 10 that which would provide illumination into the room. The fixture 10's diffuser 40 (FIGS. 3A and 3B) is removed for easier viewing of underlying structures. Visible from this view are one or more circuit boards 24 which support LED strips 26. Each LED strip 26 includes a number of white LED chips 28, which are described in detail with respect to FIGS. 2A and 2B. The size, number, and location of the LED strips 26 is variable, as are the number, type, and location of the white LEDs chips 28 on these strips. In the example shown, there are four circuit boards 24, each being approximately 1×1 foot, although a single circuit board 24 could be used as well. Although not yet shown in the figures, the circuit board(s) include a hole 25 to accommodate the fan 20.

FIG. 2A shows an example of the white LED chip 28, while FIG. 2B shows the spectrum that results from use of this chip. A white LED chip 28 is shown in top down and cross sectional views, and includes two LEDs 34a and 34b mounted to a substrate 30. A cavity wall 32 surrounds the LEDs 34s and 34b and helps to direct light out of the chip 28. Preferably, the LEDs 34a and 34b are different, and emit at different peak wavelengths. For example, LED 34a can emit at a peak wavelength of 405 nm, while LED 34b can emit at a peak wavelength of 470 nm. In this example, the LEDs 34a and 34b are covered with a phosphor 36. The phosphor 36, as one skilled in the art will appreciate, can comprise a mixture of different photosensitive chemicals. Although electrical connections to the LEDs 34a and 34b within the chip 28 are not shown, the LEDs can be driven with a current in series or in parallel, or each can be independently driven by their own currents. Each of the LEDs 34a and 34b could also be covered with their own unique phosphors as well, or covered with no phosphor at all, although this isn't shown.

As shown in the emission spectrum of the white LED chip 28 in FIG. 2B, it is assumed that the 405 nm radiation largely breaks through the phosphor 36 without being absorbed, and thus this radiation does not substantially contribute to the production of longer wavelengths which would broaden the spectrum. Thus, the spectrum shows a sharp leak at 405 nm. The 470 nm radiation by contrast is designed to interact with the phosphor 36 to produce longer wavelengths, which broadens the spectrum from about 470 to 775 nm, which in sum produces white light useful for illumination. Some amount of the 470 nm radiation is not absorbed by the phosphor 36, and thus the spectrum includes another peak at this wavelength. Thus, the overall spectrum thus has significant high intensity peaks at 405 nm and 470 nm, but also a broad spectrum that in sum produces white light. In short, the white LED chips 28 in the light box 12 produce white light having significant intensities at 405 and 470 nm. As noted above, inclusion of these peak wavelengths is preferred in the disinfecting light fixture 10 because such radiation impedes (at least) bacterial and fungal growth. One skilled in the art will understand that the disinfection benefits provided by the LEDs 34a and 34b are still had even if the peak wavelengths produced by those LEDs are not exactly at 405 nm and 470 nm. In this regard, the LEDs 34a and 34b may produce radiation at approximately 405 nm and/or 470 nm, where approximately means a wavelengths that is plus or minus 10 nm from these ideal wavelengths—i.e., from 395 nm 415 nm (in the case of the 405 nm LED 34a), and from 460 nm to 480 nm (in the case of the 470 nm LED 34b).

Further sterilization—in particular, of viruses—is provided by the UV sterilization box 14, although before discussing such details, the construction of the light fixture 10 is described, starting with FIGS. 3A and 3B. The light box 12 includes a diffuser 40, one or more circuit board sections 24 containing the LED strips 26 as already described, and a back plane 50. The diffuser 40, the circuit board(s) 24, and the back plane 50 are essentially formed in parallel planes inside the light box 12, and are held in place using a frame 36. This method of construction is described in U.S. Pat. No. 10,440,900, which was incorporated above. As explained in the '900 patent, the frame 36 can comprise four panels (for each of the four edges of the light box 12). These panels of frame 36 can be positioned around the diffuser 40, the circuit board(s) 24, and the back plane 50, and then connected to hold these structures securely in place.

The diffuser 40 is positioned between the white LED chips 28 and the room to be illuminated, and is shown in further detail in FIG. 3B. The diffuser 40 operates to scatter light produced by the white LED chips 28 to produce a combined emission spectrum (from white LED chips 28; FIGS. 2A and 2B) in the illuminated room that is more spatially homogenous. Preferably, the diffuser 40 includes a lens material 43 that is substantially transparent to the emission spectrum that the white LED chips 28 produce. The lens material 43 is typically made of various glass or plastic materials, such as a polycarbonate light-diffusing white material, and preferably allows good transmittivity of radiation at 405 and 470 nm in particular. The diffuser 40 can further include a brace 41, preferably made of a metallic material. The brace 41 acts to hold the lens material 43 and the fan grate 22, as well as providing a decorative element to the fixture 10. As shown, in this example, the brace 41 divides the diffuser 40 into quarters, and thus the lens material 43 may similarly be formed in quartered segments. Notice that the lens material 43 does not block the fan grate 22, and thus there is a hole in the lens material to allow for air flow into the light box 12 as promoted by operation of the fan 20. Note that the fan grate 22 need not be connected to the diffuser 40. In another example, the fan grate 22 can be connected to the fan 20 or to other structures in the light box 12, with the fan grate 22 then positioned in the hole in the diffuser 40 during construction.

FIGS. 3A and 3B also show details of the back plane 50. The back plane 50 is preferably formed of a single sheet of a metallic material such as steel or aluminum. The back plane 50 preferably includes a landing 58 to which the motor 44 of the fan 20 can be mounted. As best seen in FIG. 3B, the landing 58 is connected to the bulk of the back plane 50 via straps 59, thus defining holes 56 around the periphery of the landing 58. These holes 56 allow air flow to pass from the light box 12 into the UV sterilization box 14, as described subsequently. Port 89 in the back plane 50 allows for system signaling to be passed from the UV sterilization box 14 to the electronics on circuit board(s) 24 and to the fan 20, as discussed later.

As noted, the circuit board 24 can be formed in segments, and FIG. 3B shows one such segment. Notice that the circuit board segment 24 includes a cut out 25, which defines a hole when all circuit board segments are positioned in place in the fixture 10. Again, this hole 25 allows for air flow produced by the fan 20. Although such details aren't shown, the circuit board(s) 24 are preferably affixed to the back plane 50, and this can occur in different ways. The circuit board(s) 24 can be screwed to the back plane 50, possibly using stand offs which provide an air gap between the circuit board(s) 24 and the back plane 50. Alternatively, and to promote heat conduction away from the circuit board(s) 24, the circuit board(s) 24 can be affixed in good thermal contact with the back plane 50 using heat conductive tape, paste, or epoxy for example. Although not shows, the outside of the back plane 50 can include heat sinks, as explained in the above-incorporated '900 patent. Note that a benefit of incorporating fan 20 into the light box section 12 is that it promotes heat transfer away from the circuit board(s) 24, as well as air sterilization functionality.

To summarize, when the fan 20 is operating, air is drawn through fan grate 22, through the hole 25 in the circuit board(s) 24, and through holes 56 in the back plane 50 and into the UV sterilization box 14, whose construction is discussed next. As best shown in FIG. 3A, the UV sterilization box 14 includes a bottom surface 60, side surfaces 64, and a top cover 62. The inside of the UV sterilization box 14 includes baffles 70 which direct the air flow in a non-linear path and ultimately to holes 66 formed in the side surfaces 64. As noted earlier, hose connectors 16a and 16b are connected to these holes 66. As will be explained in further detail later, these baffles 70 include UV LED chips 82 to irradiate the air flow as it follows this non-linear path, which is described subsequently with respect to FIG. 4. The baffles 70 preferably comprise a metallic material, and are preferably affixed to the bottom surface 60. For example, the bottom edges of the baffles 70 can be bent 72 and affixed to the bottom surface 60 by spot welding, the use or screws, or the use of adhesives. The side surfaces 64 may be similarly attached to the bottom surface 60. In another example not shown, the baffles 70 may be integrated as a single piece, which can then be dropped into the UV sterilization box 14 during its assembly and affixed in place as necessary.

Components of the fixture 10 may be coated with antimicrobial or reflective materials. For example, the interior surfaces of the UV sterilization box 14 may be coated with Titanium Dioxide (TiO₂). As well as having antimicrobial properties, Titanium Dioxide is highly reflective, thus encouraging reflection of the UV radiation within the UV sterilization box 14. This is preferred to absorption of the UV radiation, because absorption removes useful energy that could otherwise be used for disinfection of pathogens. In one example, the coating can comprise Paint Shield®, manufactured by Sherwin Williams. Such a coating can be applied to the vertical surfaces of the baffles 70, and could also be applied to the underside of the top cover 62, and the top side of the bottom surface 60.

The top cover 62 is preferably affixed to the side surfaces 64 using screws 18. This allows the top cover 62 to be removed to perform maintenance on the fixture 10, such as to clean or remove the baffles 70 or to repair or replace system electronics, as explained subsequently. The top cover 62 can be affixed to the UV sterilization box 14 using other methods which allow it to be opened and reclosed for maintenance purposes. Although not shown, the hose connectors 16a and 16b may also connect to one or more holes provided in the top cover 62.

The UV sterilization box 14 preferably includes a safety switch 103 designed to cut power to the UV LED chips 82 when the top cover 26 is removed. This is to prevent accidental UV exposure to persons who may be assembling or maintaining the light fixture 10. This switch 103 can be provided in the UV sterilization box 14 in different ways, but as shown the switch is mounted to the top flange of the side surface 64. As one skilled will understand, switch 103 includes a contact surface that will be depressed by the top cover 62 when it is connected to the UV sterilization box 14, thus closing the switch 103 and enabling the UV LED chips 82 to receive power. When the top cover 62 is removed, the contact surface is not depressed and switch 103 is thus opened to prevent activation of the UV LED chips 82. Operation of the safety switch 103 is discussed further below with reference to FIG. 5.

The UV sterilization box 14 is preferably fully constructed and then affixed to the light box 12. In the example shown, this occurs using screws 52 which affix the bottom surface of the UV sterilization box 14 to the back plane 50 of the light box 12. However, the UV sterilization box 14 and light box 12 can be affixed using different means. Furthermore, the UV sterilization box 14 and light box 12 need not be separately constructed and then attached to each other. Instead, the fixture 10 may be constructed in a manner that integrates the functionality of the UV sterilization box 14 and the light box 12. Having said this, it can be preferable to manufacture each separately, as this makes it easier to retrofit otherwise standard light boxes 12 with a UV sterilization box 14.

As best seen in FIGS. 3A and 4, the bottom surface 60 of the UV sterilization box 14 has a hole 61 of preferably the same diameter as the hole(s) 56 formed in the back plane 50 of the light box 12, which promotes air flow from the fan 20 into the UV sterilization box 14. Once such air enters the UV sterilization box 14, it is directed through a non-linear path as directed by the positioning of the baffles 70. This is best shown in FIG. 4, which shows a top down view of the UV sterilization box 14 with the top cover 60 removed. As shown, the baffles 70 are positioned such that the air flow largely follows a serpentine path from the hole 61 in the bottom surface 60 to the holes 66 in the side surfaces 64 that meet with the hose connectors 16a and 16b. The particular manner in which the baffles 70 are positioned in FIG. 4 splits the air flow into four paths. Two of these air flow paths are shown to the right in FIG. 4, although it should be understood that two other air flow paths would be present in the left of FIG. 4, although these aren't shown for simplicity. Note that the air flow paths may not follow a strict serpentine path. For example, the baffles 70 can be positioned to create vortices 74 in the air flow paths. This effectively elongates the air flow path, which exposes air to UV radiation for a longer time, as explained further below. Baffles 72 can be positioned so as to close the air flow paths as necessary to form vortices 74, as well as to direct the air flow into the baffle structure. Note that the two air flow paths shown to the right eventually join at hole 66 to which hose connector 16b is affixed. The other two air flow paths on the left join at hole 66 to which hose connector 16a is affixed.

To more completely sterilize the air in the air flow paths, the non-linear air flow path includes UV LED chips 82, which may be formed on LED strips 80. The UV LED chips 82 and strips 80 are shown to the left in FIG. 4, although it should be understood that UV LED chips 82 and strips 80 would also be present in the right of FIG. 4, although this isn't shown for simplicity. In the example shown, the LED strips 80 are affixed to the vertical surfaces of the baffles 70, as shown in the plan view at the bottom right in FIG. 4. In this example, there are two UV LED strips 80 spaced vertically on the walls of the baffles 70, which improves exposure of the air to UV radiation.

Preferably, as much of the non-linear air flow paths are exposed to UV radiation as possible, and so in FIG. 4 the UV LED strips 80 are essentially positioned along the entirety of the lengths of the air flow paths, and further preferably are positioned along at least half of these lengths. The width d of the air flow paths around the baffles 70 can may be approximately 1 to 1.5 inches. Assuming that the UV sterilization box 14 is approximately 1.5×1.5 feet (X2, FIG. 1), the length of each of the four air flow paths is approximately 60 to 100 inches, and thus irradiation preferably occurs for at least approximately 30 to 50 inches along these paths. Because the UV radiation may be harmful to people, it is preferable that the UV LED strips 80 not appear in positions where the UV radiation could shine or leak out of the UV sterilization box 14. Thus, for example, the UV LED strips 80 are not proximate the air input hole 61, nor are they proximate the output holes 66 to which the hose connectors 16a and 16b are affixed. UV LED strips 80 may as shown be placed on both sides of the baffles 70, which irradiates the air flow paths from opposing sides. While it is preferred to place the UV LED strips 80 on the vertical surfaces of the baffles 70, they could be placed elsewhere as well, such as on the top side of the bottom surface 60, or the underside of the top cover 62.

Assuming that the height of the UV sterilization box 14 is about 4.5 inches (H2, FIG. 1), the total volume of each of the four air flow paths is approximately 360 cubic inches. Fan 20 may for example comprise Part No. 09225VA-12K-AA-cc, manufactured by NMB Technologies Corp., which moves air with a flow rate of 54 cubic feet/minute, which would move air through each of the four air flow paths in parallel at a flow rate of 13.5 cubic feet/minute, or 389 cubic inches/second. As such, each unit volume of air in each flow path is constantly UV irradiated for approximately one second (360/389), and with a high flux or energy density because the air is being irradiated almost continuously along the length of each air flow path. Note this is advantageous when compared with other air purification system that use UV radiation to purify air. Typically, such systems involve a point UV source which the air to be sterilized rushes passed, meaning that each unit volume of air is only radiated for a short time, which may result in incomplete inactivation of pathogens. By contrast, the air is constantly irradiated in the UV sterilization box 14 along the non-linear paths for an extended period of time, and with a high flux or energy density, thus ensuring more complete disinfection. Of course, the extent to which air is UV irradiated could be varied by changing the flow rate of the fan 20, changing the length or volume of the air flow paths, changing the intensity and number of UV LED chips 82 used, etc.

In one example, each of the UV LED chips 82 on UV LED strips 80 produces UV radiation with a peak wavelength in the range of 200 to 280 nm, which generally corresponds to the range of UV-C wavelengths. More preferably, the UV radiation has a peak wavelength in the range of 240 to 260 nm, or in the range of 260 to 280 nm. UV radiation in this range has been shown to be particularly useful to inactivate viruses by targeting their nucleic acids. See K. Bergmann, “UV-C Irradiation: A New Viral Inactivation Method for Biopharmaceuticals,” America Pharmaceutical Review, Vol 17(6) (November 2014).

While FIG. 4 shows four air flow paths each following a non-linear path, and two output holes 66, it should be understood that this is just one example. There could be more or less air flow paths established with the UV sterilization box 14, or more or less holes 66. For example, a single non-linear path could comprise a spiral in which air input via hole 61 spirals around the box 14 at increasing diameters, until the sterilized air eventually exits the box at a single output hole 66.

FIG. 4 shows further options that can be included with the UV sterilization box 14, and in particular with the hose connectors 16a and 16b. As shown in the upper right, the interior diameter of the hose connectors 16a/b includes a one-way valve 93 that only allows sterilized air to pass out of the UV sterilization box 14. The hose connectors 16a/b may also include a pressure relief valve 95 which is designed to vent the sterilized air should it exceed the valve 95's pressure. The interior diameter of the hose connectors 16a/b can also include filters 97, such as charcoal filters, to further filter particulates and pathogens, and to also work as an anti-odorant. The anti-odorant properties of the filter 97 can be particularly useful when the fixture 10 is used in a grow farm setting and when the plants being grown have strong odors (e.g., cannabis). The filters and valves need not necessarily be positioned within the hose connectors 16a/16b, but could comprise discrete components connected to the hose connectors 16a/b outside the box 14. Although not shown, the air flow paths within the UV sterilization box 14 could include filters and valves at various points as well.

As shown in FIG. 4, the UV sterilization box 14 can include an electronics section 15. This section 15 can be walled off from the baffles 70 and the air flow paths by a wall 90. Section 15 can include the driver circuitry 92a for driving drive the white LED chips 28 in the light box 12 and driver circuitry 92b for driving the UV LED chips 82 in the UV sterilization box 14. It is preferable that the driver circuits 92a and 92b be separate because the white LED chips 28 and UV LED chips 82 may have different driving requirements (voltages, currents, power, etc.). Driver circuitries 92a and 92b could also be integrated in another example.

Electronics section 15 can include or more ports 86 which receive AC power 100 (FIG. 5) from outside the fixture 10, e.g., from a socket or other power source or line to which the fixture 10 is connected. The section 15 may also include a port 88 in the bottom surface 60 to allow signaling to be output from driver circuitry 92a to the white LED chips 28 in the light box 12. Port 88 can correspond in position to a similar port 89 in the back plane 50 of the light box 12 (see FIG. 3B). Although not shown, one skilled will understand that such signaling will connect to connectors or contacts on one or more of the circuit board(s) 24. AC power for the fan 20 can also pass through the ports 88/89.

Electronics section 15 may also include one or more ports 84 to allow signaling to be output from driver circuitry 92b to the UV LED chips 82 in the UV sterilization box 14 and to the safety switch 103. One skilled will understand that such signaling will connect to each of the UV LED strips 80. In this regard, it can be useful to connect the various UV LED strips 80 within the UV sterilization box in a manner to reduce the amount of signaling and connections required. Although not shown, the bottom surface 60 can include a circuit board to assist in routing signaling to the UV LED strips 80. Preferably, port(s) 84 are optically blocked after the signaling has passed through to prevent UV light from entering electronics section 15. It is preferable to include the system electronics within section 15 so it can be easily accessed. For example, top cover 62 of the UV sterilization box 14 can be removed (using screws 18, FIG. 3A), thus allowing access as necessary to maintain or replace system electronics. System electronics could also be located in the light box 12. The size of electronic section 15 can vary depending on the size of the system electronics that are supported.

System electronics are shown in FIG. 5. AC power provides a voltage Vac, which is provided to the white LED driver circuitry 92a, to the UV LED driver circuitry 92b, and to the fan 20. Although not shown, it should be understood that Vac may be processed (transformed, rectified to DC voltages, etc.) prior to being provided to the driver circuitries 92a and 92b and fan in accordance with their input power needs. White LED driver circuitry 92a typically provides a compliance voltage Vw as necessary to provide a current Iw necessary to drive the white LED chips 28. A regulator 94a can be used to control Iw, as is well known. UV LED driver circuitry 92b is similar, and provides a compliance voltage Vuv as necessary to provide a current Iuv necessary to drive the UV LED chips 82, with a regulator 94b controlling Iuv. In one example, the power required by the fixture 10 may comprise about 100 Watts, with the white LED chips 28 requiring about 60 W, the UV LED chips 82 requiring approximately 30 W, and the fan requiring about 10 W.

It may be desired to separately control one or more aspects of the fixture 10. For example, it may be desired at a given time to drive only the white LED chips 28 to provide illumination to a room the fixture 10 is placed in, but to not drive the UV LED chips 82 to provide UV disinfection. Conversely, it may be desired at a given time (e.g., at night) to drive only the UV LED chips 82 to provide UV disinfection, but to not drive the white LED chips 28 to provide illumination. In this regard, the fixture 10 can include or be controlled by one or more switches 100, 102, or 104. For example, switch 100 comprises a master switch used to control all operations of the fixture, i.e., to control driving the white and UV LED chips 28 and 82, and the fan 20. Switch 102 can be used to independently control the white LED chips 28. Switch 104 can be used to independently control the UV LED chips 82 and the fan 20. Switch 104 is useful because it would normally be expected that the fan 20 and UV LED chips 82 would be enabled together, with the fan 20 drawing air flow into the UV sterilization box 14 that includes the chips 82. That being said, the UV LED chips 82 and fan 20 could also be independently controlled by their own switches. Any of the switches shown could comprise wall-mounted switches to which the fixture is connected. Alternatively, the switches can appear in the light fixture (section 15) as part of the system electronics. In this respect, the switches may be controlled by a remote control, with system electronics including a wireless receiver (e.g., a Bluetooth receiver) for receiving input from the remote control.

System electronics can further include a safety switch 103. As described earlier, this switch 103 is designed to open to cut power to the UV LED chips 82 (e.g., via driver circuitry 92b) when the top cover 62 is removed from the UV sterilization box 14. As shown, safety switch 103 is in series with switch 104, and so would also disable power to the fan 20. However, switch 103 could also be located in the circuitry to cut power to only the LED driver circuitry 92b.

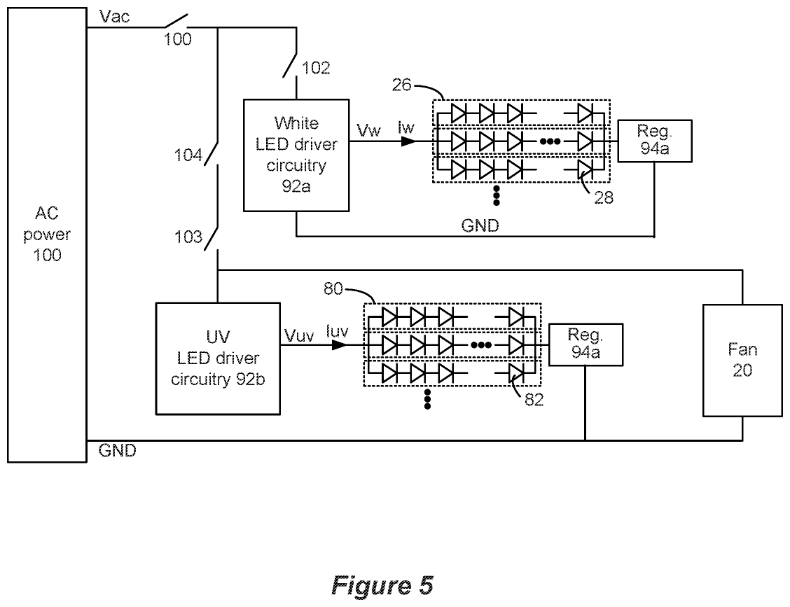

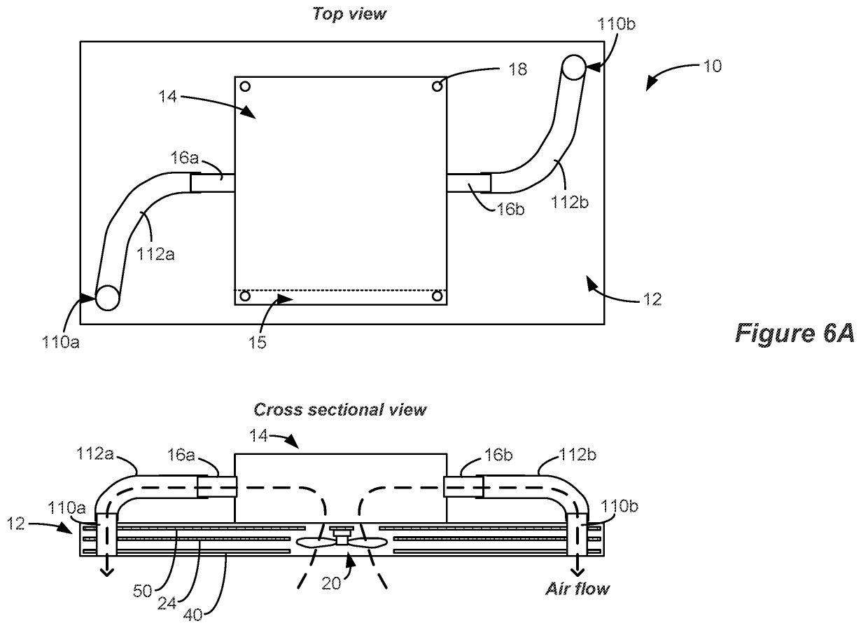

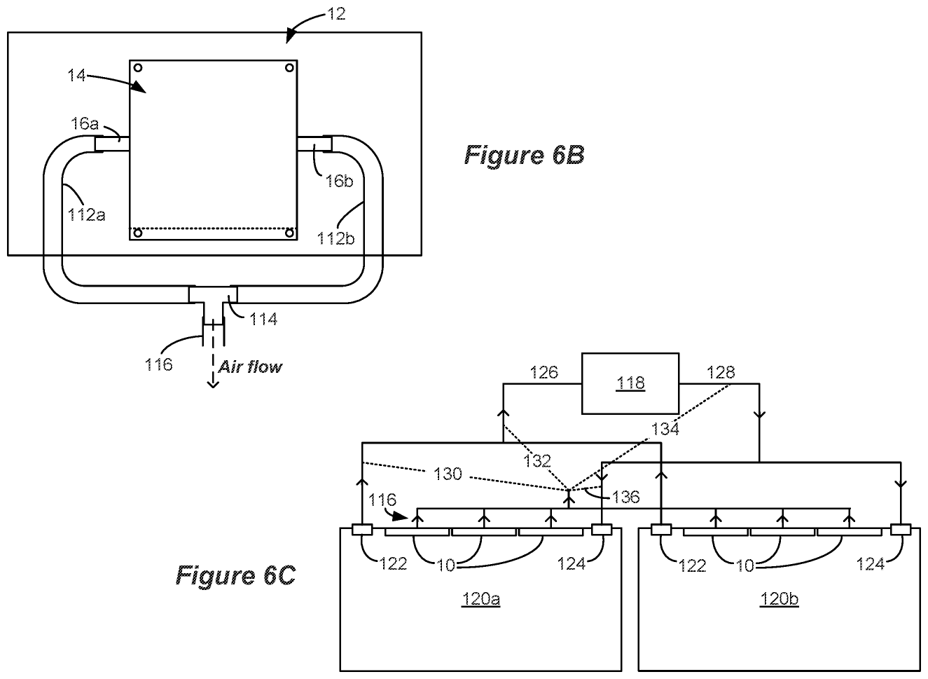

As discussed above, the UV sterilization box 14 includes one or more hose connectors 16a and 16b which output sterilized air, and such sterilized air is preferably distributed back into the room or building in which the fixture 10 appears. FIGS. 6A-6C show different examples of how this can occur. Sterilized air can also effectively be disposed with, such as by venting such air into the plenum space in a building or house, or through a vent to the outside environment.

FIG. 6A shows an example in which the sterilized air is output back into the room through the fixture 10 itself. In this example, which shows a larger light fixture (2×4 foot), the light box 12 includes one or more hose ports. Two such ports 110a and 110b are shown in FIG. 6A, and may comprise hose connectors allowing them to be joined to the hose connectors 16a and 16b by hoses 112a and 112b as shown. The ports 110a and 110b in this example proceed through holes in the back plane 50, the circuit board(s) 24, and the diffuser 40 of the fixture 10. Although not shown, the air output from the hose connectors 16a and 16b can be combined (e.g., FIG. 6B) and put back into the room through a single port 110 in the light box 12, or through more than two ports.

FIG. 6C shows that the air output from the hose connectors 16a and 16b can be placed into the air handling system in a building in which one or more fixtures 10 are placed, thus providing sterilized air to one or more rooms in the building. In this example, it is assumed that the building has a number of rooms (two of which 120a and 120b are shown) with each room having a number of fixtures 10 (three in each as shown). The building includes an air handler 118 with an input 126 and an output 128. One skilled will recognize that the duct work of an air handling system could include other components that are not shown, such as fans, exhaust vents, fresh air inputs, etc. Each room 120a and 120b has a supply vent 124 connected to the output 128 and a return vent 122 connected to the input 126. FIG. 6B shows that the air output from the hose connectors 16a and 16b in a given fixture 10 can be combined (e.g., FIG. 6B) using a junction 114, which outputs to an output hose 116. Junction 114 and output hose 116 could also be fit with filters (97) and valves (93, 95), as explained earlier with reference to FIG. 4. The outputs from several output hoses 116 can be connected as shown in FIG. 6C, and connected by another hose or duct work to any convenient point in the air handler duct system, including the return line of a given room (130), the input 126 to the air handler 118 (132), the output of the air handler 118 (134), or to the supply line of a given room (136). In any of these examples, the sterilized air is ultimately provided back into the room(s).

Many modifications to the disclosed fixture 10 can be made, and the fixture 10 can be used in different environments to useful ends. For example, the white LED chips 28 may not include significant peaks at either or both of 405 nm or 470 nm, although the inclusion of these wavelengths is preferred to further aid sterilization that the fixture 10 provides. In fact, the white LED chips 28 may not be used, and instead other white light sources (e.g., bulbs) could be used in the fixture 10, with disinfection occurring strictly through use of the fan 20 and the UV sterilization box 14. The UV sterilization box 14 could include UV radiation sources other than UV LED chips. For example, various UV emitting bulbs could be used inside the UV sterilization box 14.

The fixture 10 can be used in environments where pathogens may be present, and in particular air borne pathogens. This can include hospitals, nursing homes, operating rooms, restrooms, kitchens, etc. Fixture 10 can also be used in a grow farm setting, in which light fixtures 10 are used to grow plants. For example, the disclosed fixture can be used in the context of the above-incorporated '900 patent, and can include the various improvements to a light fixture that are disclosed in that document.

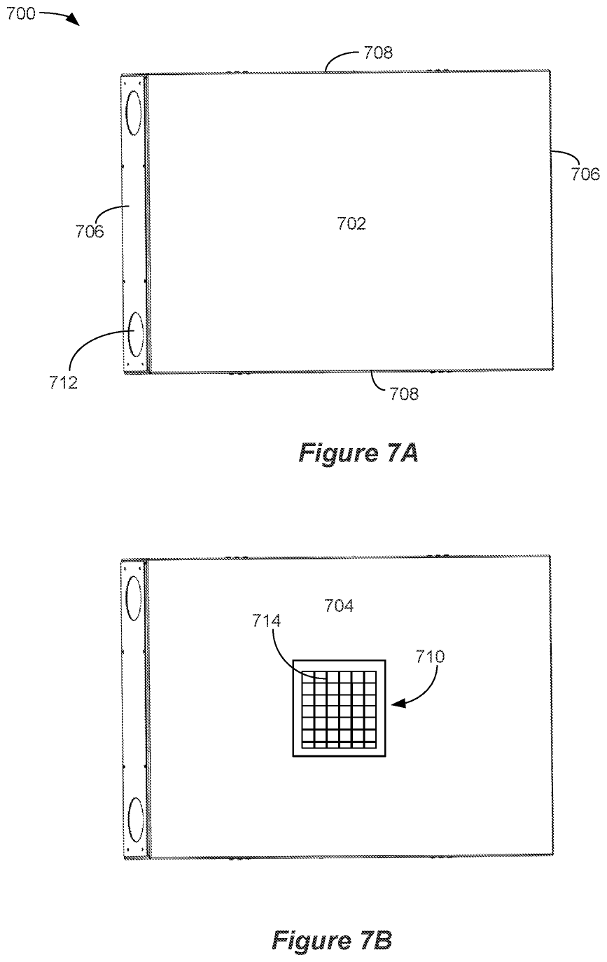

FIGS. 7A and 7B show perspective views of another embodiment of a sterilization box 700 from the top (FIG. 7A) and from the bottom (FIG. 7B). The sterilization box comprises a top 702, a bottom 704, two ends 706, and two edges 708. The sterilization box comprises an intake port 710 configured within the bottom 704. The illustrated embodiment is configured with four output ports 712. In the illustrated embodiment two output ports 712 are configured in each of the two ends (though the output ports in one of the ends is not visible in the drawing). However, other embodiments may comprise more or fewer output ports. The output ports may be configured with hose connectors (not shown) as illustrated in FIG. 1.

As with the embodiments described above, the sterilization box may be configured to mount in the ceiling of a room, so as to treat room air. The bottom of the sterilization box 700 may be configured to attach to a light box, as described above. Alternatively, the bottom of the sterilization box 700 may be configured to attach to a ceiling tile. Accordingly, the sterilization box may be sized and configured to interface with a 2×2 feet or 2×4 feet fixture or ceiling tile, as described above. The illustrated embodiment 700 is most ideally configured to attach to a 2×2 fixture or tile. When attaching to a ceiling tile, the input port (or an extension thereof) may be configured to protrude through the ceiling tile to draw in air from the room. Air drawn into the sterilization box is treated as described in more detail below and may be put back into the room in which the sterilization box is placed. According to some embodiments, the sterilization box may be equipped with hoses and hose ports for directing the treated air back into the room, as illustrated for the previously described fixtures in FIG. 6A. According to some embodiments the treated air may be reintroduced into the room at a distance of at least several feet from the sterilization box intake so as to facilitate circulation of treated air throughout the room. The sterilization box may be configured for other mounting options. For example, the sterilization box may be mounted to any surface, for example, a ceiling or the like.

A difference between the sterilization box 700 and the sterilization boxes described earlier (e.g., sterilization box 14, FIG. 1) resides in how room air is drawn into the box. Recall that the fixture 10 illustrated in FIG. 1 features a central fan 20 that forces air into the sterilization box and pushes the air through the box until the treated air is pushed out of the sterilization box via the holes 66 (FIG. 3A). Accordingly, the sterilization box 14 operates at a slight overpressure with respect to the atmosphere of the room. By contrast, the sterilization box 700 does not feature a centralized fan at the input port 710. Instead, fans are located at the output ports, as shown in more detail below. The fans at the output ports create a slight under-pressure that draws room air into the sterilization box via the input port. The inventors have determined that in some embodiments, using output fans to create negative pressure in the sterilization box provides more effective airflow and sterilization in the box. The input port 710 may be equipped with an intake filter or mesh 714, which may be photocatalytically active, as described below.

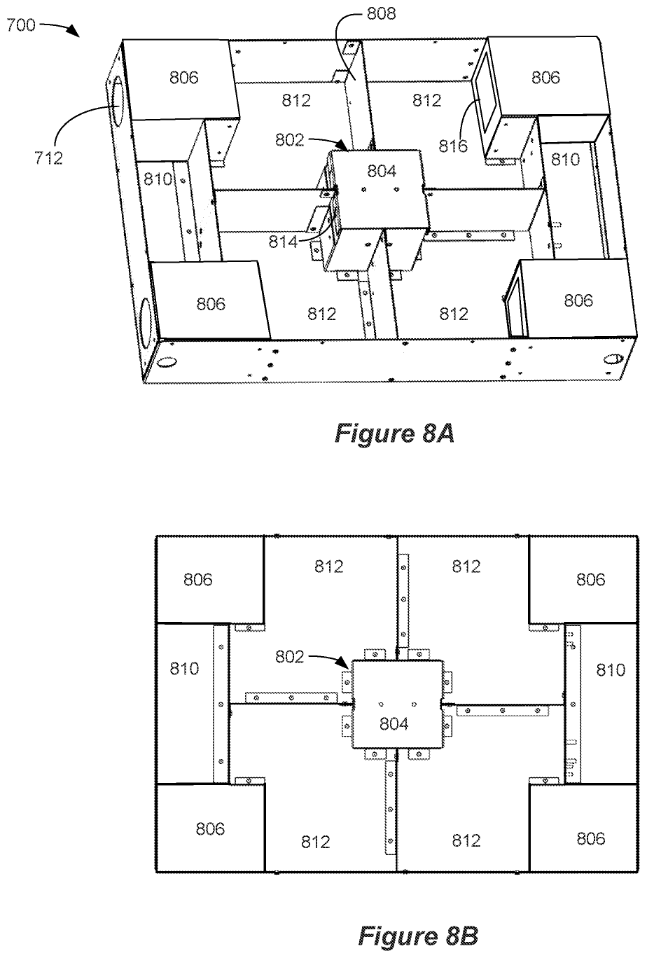

FIGS. 8A and 8B show top views of the sterilization box 700 in perspective and plan view, respectively, from above with the top (702, FIG. 7A) removed. The sterilization box comprises an intake chamber 802 having a top 804 and four walls (not numbered). Room air enters the intake chamber 802 from the bottom via the intake port 710 (FIG. 7B). The illustrated sterilization box has four fan boxes 806. Depending on the configuration of the particular embodiment, more or fewer fan boxes may be included. The fan boxes each comprise a fan configured to move air out of the sterilization box through the output ports 712. The fan boxes may also include a filter, such as a HEPA and/or activated charcoal filter configured to filter the air as it enters and exits the sterilization box.

Internal walls, such as wall 808 may be used to define various spaces within the sterilization box. For example, electronics spaces 810 may be defined. The electronics spaces may contain circuitry for powering and driving the fans, the LEDs, etc., as described above. The sterilization box also comprises flow spaces 812. The illustrated embodiment comprises four flow spaces 812, but various embodiments may comprise more or fewer flow spaces (i.e., one or more flow spaces). In operation, fans within the fan boxes create negative pressure created within the sterilization box, which draws air into the intake chamber 802 via the intake port 710 (FIG. 7). Air is first treated in the intake chamber (described further below) and then drawn out of the intake chamber and into the flow spaces 812 via windows 814 in the intake chamber. The flow spaces 812 are configured to include baffles (not shown), which impede/slow the flow of as it is further treated in the flow spaces 812, as described in more detail below. Air is then drawn from the flow spaces into the fan boxes 806 via fan box windows 816. The treated air is then reintroduced to the room via output ports 712 and hoses and hose ports, as described above.

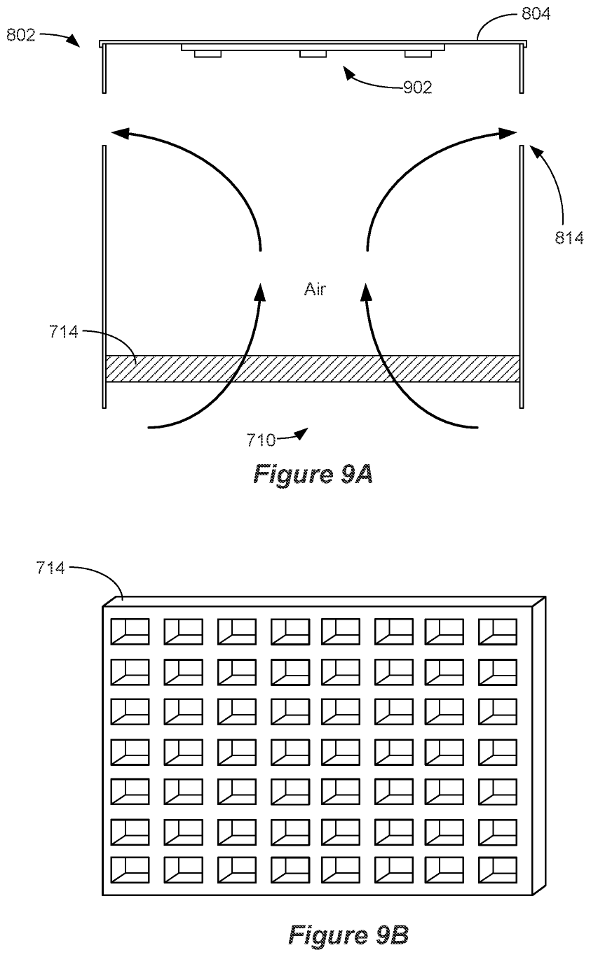

FIG. 9A shows a cross section of the intake chamber 802. As shown, air is drawn into the intake chamber 802 via the intake port 710 through the intake filter 714, which is shown in more detail in FIG. 9B. The intake filter 714 may comprise photo-catalytically active material, such as TiO₂, zinc oxide (ZnO), tungsten oxide (WO₃), or the like. According to some embodiments, the intake filter comprises anatase, or anatase-rutile mixtures of TiO₂. The intake chamber is configured with LEDs 902, which may be mounted to the inside of the top 804 of the intake chamber. According to some embodiments the LEDs may emit light in the UV-A region of the UV spectrum (315-400 nm). According to some embodiments, the LEDs may comprise bare diodes without any wavelength-converting phosphor. One example of suitable UV-A LEDs is part number NZ5-CUN66B1G, available from Seoul Viosys (Seoul, South Korea).

The emitted UV-A radiation may disinfect biological agents in the air directly. Also, the UV radiation may interact with the photocatalytic material of the intake filter 714 to open other pathways for the treatment of biological and other organic species, such as VOCs in the air. For example, some of the biological and/or VOC contaminants may adsorb on the intake filter and become oxidized by photocatalytically generated charge carriers on the filter's surface. Also, the UV illumination of the filter material may generate reactive oxygen species (ROS), such as superoxide anions, hydroxyl radicals, and ozone, which may oxidize surface-adsorbed and airborne biological and/or VOC contaminants within the intake chamber. Air exits the intake chamber via windows 814.

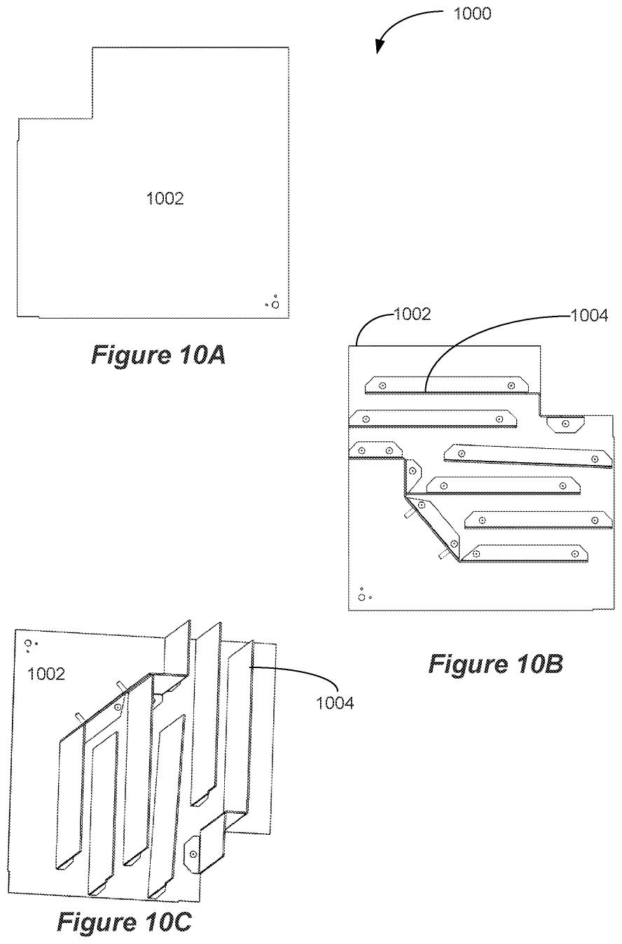

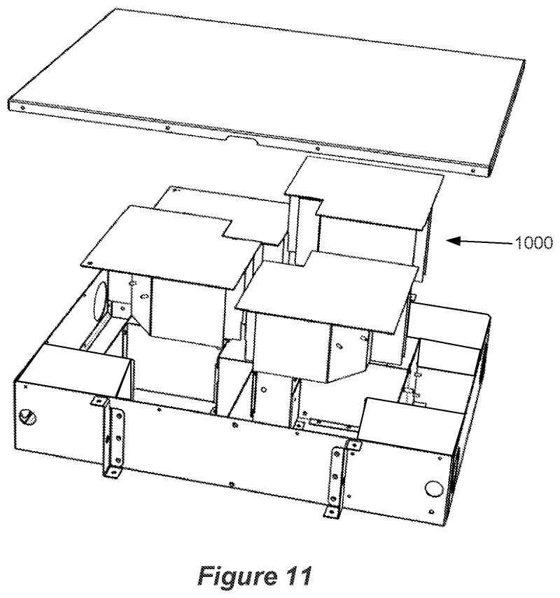

As explained above, air that is first treated in the intake chamber 802 passes from the intake chamber to the flow spaces 812 (FIGS. 8A and 8B). As also mentioned above, the flow spaces may be configured with baffles that crate a serpentine flow path that slows/impedes the flow of air therethrough. FIGS. 10A, 10B, and 10C show top, bottom and perspective views, respectively, of an embodiment of a baffle insert 1000 that can be inserted into each of the flow spaces 812. The baffle insert 1000 comprises a top plate 1002 with baffles 1004 affixed thereto. FIG. 11 shows an exploded view of the sterilization box 700 showing how the baffle inserts 1000 fit into the flow paths.

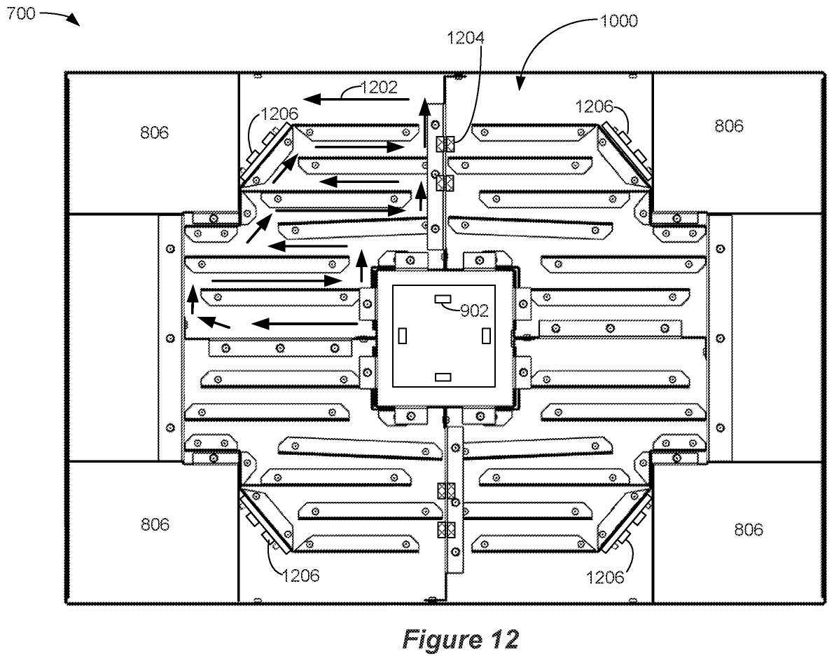

FIG. 12 shows the sterilization box 700 from the bottom with the bottom cover 704 (FIG. 7B) and the intake filter 714 (FIG. 7B) removed. The baffle inserts 1000 are installed within the flow spaces 812 (FIGS. 8A and 8B). The arrows 1202 illustrate the air flow through one of the flow spaces. LEDs 1204 are configured to irradiate the air as it makes its way through the serpentine flow path. It should be noted that the LEDs 1204 may be located at other positions within the flow spaces than the positions illustrated. According to some embodiments, the LEDs 1204 may be configured to emit light in the UV-C region of the spectrum (e.g., 200-280 nm), which is primarily active for disinfecting biological species in the air, as described above. The UV-C LEDs may be bare diodes that do not include wavelength-converting phosphor. One example of suitable UV-C LEDs is part number CUD7W9560A, available from Seoul Viosys (Seoul, South Korea). The flow path of the sterilization box may be configured with additional UV-A emitting LEDs (in addition to the UV-A LEDs 902, discussed above). For example, the illustrated sterilization box has UV-A LEDs 1206 positioned to irradiate the air as moves from the flow spaces into the fan boxes 806.

As explained above, the inside parts of the sterilization box may be painted, coated, powder coated, etc., with TiO₂— containing material, which (1) causes the inside of the box to be highly reflective, thereby maximizing exposure of the air to multiple reflections of disinfecting light, and (2) acts as a photocatalyst to increase the disinfection. According to some embodiments, the baffles are configured to facilitate vortices in the air flow paths, as described above. For example, notice in the illustrated embodiment that the baffles are not perfectly parallel to each other. This causes the flow path to narrow and widen, which promotes non-uniform air flow. This increases the amount of time the air is exposed to disinfecting UV radiation.

As mentioned above, the embodiment of the sterilization box 700 illustrated in FIGS. 7-12 is ideally configured to attach to a 2×2 foot ceiling fixture or ceiling tile. However, other sizes and configurations are contemplated by the disclosure. For example, another embodiment (not illustrated) comprises a sterilization box configured to attach to a 2×4 foot tile or fixture. Some 2×4 foot embodiments may comprise an intake chamber, as described above, and two flow spaces having serpentine flow paths. Such an embodiment may comprise two fans and two output ports.

According to some embodiments, the sterilization boxes described herein may be used in conjunction with a sensor system, such as the system described in U.S. patent application Ser. No. 17/317,656, (“the '656 application”) filed May 11, 2021, the entire contents of which are hereby incorporated herein by reference. One or more sterilization boxes may communicate with a sensor module, which may be configured to sense different environmental conductions. The conditions may be provided to the sterilization box and a control algorithm may use the sensed conditions to control one or more functions of the sterilization box, such as illumination provided by the various LEDs (e.g., the UV-A and/or UV-C LEDs), fan speed, etc. According to some embodiments, a room or building may be equipped with more than one sterilization boxes. In such situations, one sterilization box may be the master or control box and others of the boxes may be designated as daughter boxes. The master or control box may output necessary control signals to the daughter boxes based on data sensed using the sensor system. The various sensor modules and sterilization boxes may communicate using a wireless network, such as a mesh network, for example. The control and communications hardware may be configured within the electronics spaces 810 (FIG. 8) of the sterilization boxes.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An air sterilization box for treating room air, the sterilization box comprising:
an intake chamber configured to receive room air drawn into the sterilization box,
a first plurality of first ultraviolet (UV) light emitting diodes (LEDs) within the intake chamber configured to irradiate air drawn into the intake chamber with UV radiation having a first peak wavelength within a first wavelength range,
one or more flow paths configured to receive air from the intake chamber, and
a second plurality of second UV LEDs within the one or more flow paths configured to irradiate air drawn through the flow paths to produce treated air, wherein the second UV LEDs provide radiation having a second peak wavelength that is different than the first peak wavelength.

2. The sterilization box of claim 1, wherein the first UV LEDs are configured to produce UV radiation with a peak wavelength in the range from 315 to 400 nm.

3. The sterilization box of claim 1, wherein the intake chamber comprises a photoactive filter comprising titanium dioxide (TiO₂) configured to catalyze generation of reactive oxygen species (ROSs) when irradiated with radiation from the first UV LEDs.

4. The sterilization box of claim 3, wherein the intake chamber is configured so that the ROSs oxidize volatile organic compounds (VOCs) in air drawn into the intake chamber.

5. The sterilization box of claim 1, wherein the chamber comprises one or more windows configured to pass air in the intake chamber into the one or more flow paths.

6. The sterilization box of claim 1, wherein each of the flow paths terminate at an exit port and wherein the sterilization box further comprises a fan at each exit port configured to move treated air out of the sterilization box, thereby creating an under-pressure within the sterilization box.

7. The sterilization box of claim 1, wherein each of the one or more flow paths are non-linear.

8. The sterilization box of claim 7, wherein each of the flow paths are serpentine or spiral along at least a portion of their lengths.

9. The sterilization box of claim 8, wherein each of the flow paths are defined by one or more baffles.

10. The sterilization box of claim 9, wherein each of the flow paths have widths that change over the length of the flow path.

11. The sterilization box of claim 1, wherein the second peak wavelength in the range from 200 to 280 nm.

12. The sterilization box of claim 1, wherein the second UV LEDs are configured to produce the UV radiation capable of sterilizing biological pathogens in the air.

13. The sterilization box of claim 1, wherein the sterilization box comprises an interior comprising a reflective coating.

14. The sterilization box of claim 13, wherein the reflective coating is photocatalytically active.

15. The sterilization box of claim 14, wherein the reflective coating comprises TiO₂ crystals.

16. The sterilization box of claim 1, further comprising a bottom configured to connect to a light fixture.

17. The sterilization box of claim 1, further comprising a bottom configured to connect to a ceiling tile.

18. The sterilization box of claim 1, comprising four flow paths.

19. The sterilization box of claim 1, comprising two flow paths.

20. The sterilization box of claim 1, further comprising filters at each of the exit ports configured to filter the treated air as it exits the sterilization box.
